(19) United States
(12) Patent Application Publication
(10) Pub. No.: US 2018/0057847 A1
(43) Pub. Date: Mar. 1, 2018

(54) Silicone Oil as Reaction or Separation Medium for Ester Synthesis

(71) Applicant: Biosolutions USA, LLC, Reno, NV (US)

(72) Inventors: David Lee Bernick, Ben Lomond, CA (US); Wade Morgan Dugdale, Santa Cruz, CA (US); Stanislav Fridland, San Francisco, CA (US)

(21) Appl. No.: 15/691,671

(22) Filed: Aug. 30, 2017

Related U.S. Application Data
(60) Provisional application No. 62/381,493, filed on Aug. 30, 2016.

(51) Int. Cl. C12P 7/62; C12P 7/16

(52) U.S. Cl. CPC C12P 7/62; C12P 7/16

(57) ABSTRACT

Silicone oil is a reaction medium or separation medium, or both, for ester synthesis. Enzyme-catalyzed esterification is conducted in the silicone oil, with ester product soluble therein. Ester is separated from silicone by distillation. Esterification in silicone oil in conjunction with a simultaneous fermentation process, results in alcohol fermentation products flowing through the intervening silicone oil (serving as an oxygen barrier) to react with organic acid at an oil:aqueous interface, catalyzed by an enzyme (e.g., lipase, esterase). Ester solubility in silicone is leveraged for extraction, isolating catalyzed (metal, chemical or enzyme) ester reaction products from an original reaction (aqueous or organic) medium. Esters may be generated from alcohol fermented from organic matter of agricultural waste products, utilizing cheaply available silicone oil. Similarly, silicone oil may also be used to extract from a polar solvent (e.g., water), medium chain length alcohols during synthesis performed by a microorganism.

### CROSS-REFERENCE TO RELATED APPLICATIONS

This patent application claims the benefit of U.S. patent application 62/381,493, filed Aug. 30, 2016, which is incorporated by reference along with all other references cited in this application.

### BACKGROUND OF THE INVENTION

Synthesis of ethyl-acetate (ethyl-ethanoate, AcOEt), ethyl-lactate (4-hydroxy ethylpropanoate, LacOEt), and other esters, can be accomplished by the reaction of an alcohol and an organic acid. Typically these reactions are carried out in acidic, aqueous conditions, where the reaction (condensation) produces the desired ester product, as well as water (H20).

Under certain conditions the esterification reaction is acid catalyzed. The acid catalyzed reaction is known as Fisher-Speir esterification.

The esterification reaction is reversible, such that the ester bond that is formed can be hydrolyzed (i.e., subject to hydrolysis) and re-converted back into the initial alcohol and organic acid. In fact, one challenge encountered during esterification is a need to rapidly isolate the ester product from water. Under aqueous conditions (and especially with low molecular weight esters such as AcOEt and LacOEt), such hydrolysis of the product back to its component parts can happen rapidly, degrading reaction yield.

Accordingly, there is a need in the art for improved approaches for the synthesis of esters.

### BRIEF SUMMARY OF THE INVENTION

Embodiments utilize silicone oil as a reaction medium or separation medium, or both, for ester synthesis. Certain embodiments may conduct an enzyme-catalyzed esterification reaction in the silicone oil, with the ester product being soluble therein. The ester product may subsequently be separated from the silicone oil by simple pot distillation techniques. Particular embodiments may employ an esterification reaction in silicone oil in conjunction with a simultaneous fermentation process. There, alcohol fermentation products may flow through the intervening silicone oil (serving as an oxygen barrier) to react with organic acid at an oil:aqueous interface, catalyzed by an enzyme (e.g., lipase or esterase). Alternative embodiments may leverage ester solubility in silicone oil for extraction purposes, thereby isolating catalyzed (metal, chemical, or enzyme) ester reaction products from an original reaction (aqueous or organic) medium. Embodiments may be particularly suited to generate large quantities of esters from alcohol fermented from organic matter of agricultural waste products (e.g., plant husks), utilizing cheaply available silicone oil.

In an implementation, a method includes conducting a reaction between an alcohol and an organic acid to produce an ester, and dissolving the ester in a silicone oil. The reaction can be conducted in the silicone oil. The method can further include catalyzing the reaction with an enzyme. The enzyme can be produced by a halophilic organism. The enzyme can be Lipase B. The enzyme can be immobilized. The enzyme can be immobilized at an interface between the silicone oil and an aqueous mixture including the organic acid.

When the reaction is conducted in the silicone oil, the method can further include removing the silicone oil including the dissolved ester, and replenishing fresh silicone oil. When the reaction is conducted in the silicone oil, the method can further include simultaneously performing a fermentation process to produce the alcohol. Further, the fermentation process can be simultaneously performed in a same reaction vessel containing the silicone oil, and the alcohol flows into the silicone oil to drive the reaction. The fermentation process can utilize an agricultural waste product as a starting material. The fermentation process can be a mixed fermentation process also producing the organic acid.

The ester can be dissolved in the silicone oil by an extraction process. The reaction can be catalyzed by an acid. The reaction can be catalyzed by an enzyme. The method can further include separating the ester from the silicone oil by distillation. The reaction can occur in an emulsion comprising the silicone oil and an aqueous phase including the organic acid.

In an implementation, a method includes: conducting a microbial fermentation reaction to generate a medium chain length alcohol; dissolving the medium chain length alcohol in a silicone oil; and removing the medium chain length alcohol from the silicone oil by distillation. The method can further include following removal of the medium chain length alcohol from the silicone oil, circulating the silicone oil back to the microbial fermentation reaction to dissolve additional quantities of the medium chain length alcohol. The medium chain length alcohol can include butanol.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIG. 1 shows a simplified view of a reaction environment according to an embodiment.

FIG. 2 shows a simplified flow diagram of a method according to an embodiment.

FIG. 3 shows a simplified view of a reaction environment according to an alternative embodiment.

### DETAILED DESCRIPTION OF THE INVENTION

Esterification reaction mechanisms catalyzed by enzymes offer an alternative to acid-catalyzed approaches. For example, the esterification reaction can be catalyzed using a hydrolase enzyme of the lipase or esterase families. According to the nomenclature established by the International Union of Biochemistry and Molecular Biology, these enzymes are classified as E.C 3.1.1.x.

Under aqueous conditions, certain enzymes have preference for the hydrolysis reaction. For example, the enzyme Lipase B (also known as CalB) from the yeast species Candida antartica, is classified as a member of E.C 3.1.1.3 (Lipase). Lipase B is known to hydrolyze the ester bonds in triacylglycerols, yielding a diacylglycerol and a carboxylate (salt of an organic carboxylic acid).

Enzymes may also exhibit activity in nonaqueous environments. For example, the CalB enzyme is functional in diethyl ether solvent, catalyzing the acetyl transfer from AcOEt to an amine. Enzymatic activity may also be present in other nonaqueous solvents, including but not limited to, heptane, hexane, toluene, acetone, chloroform, and acetonitrile.

Silicone oils are polymerized siloxanes, functionalized with organic side chains. Two classes of these silicone oils include dimethyl and methyl-phenyl derivatives.

Silicone oil polymers can be linear or cyclic. Common linear forms of silicone oils include polydimethyl siloxane (PDMS) and polymethylphenylsiloxane (PMPS). The cyclical forms are named cyclosiloxanes.

Silicone oils are typically fairly inert, offering desirable properties such as high temperature stability and low flammability. Thus, one common use of silicone oils is as brake fluids in automobiles. This and other existing applications for silicone oils result in their being widely available at relatively low cost.

Moreover, silicone oils are offered by manufacturers in a variety of different viscosities and molecular weights. Certain low polarity organic molecules, including many esters, are miscible or soluble in silicone oils.

Accordingly, embodiments utilize silicone oil as a reaction medium or separation medium, or both, for ester synthesis. Certain embodiments may conduct an enzyme-catalyzed esterification reaction in the silicone oil, with the ester product being soluble therein. The ester product may subsequently be separated from the silicone oil by simple pot distillation techniques. Particular embodiments may employ an esterification reaction in silicone oil in conjunction with a simultaneous fermentation process. There, alcohol fermentation products may flow through the intervening silicone oil (serving as an oxygen barrier) to react with organic acid at an oil:aqueous interface, catalyzed by an enzyme (e.g., lipase or esterase). Alternative embodiments may leverage ester solubility in silicone oil for extraction purposes, thereby isolating catalyzed (e.g., acid, metal, or enzyme) ester reaction products from an original aqueous or organic medium. Embodiments may be particularly suited to generate large quantities of esters from alcohol fermented from organic matter of agricultural waste products (e.g., plant husks), utilizing silicone oil cheaply available for other applications.

Various embodiments may employ silicone oil as a nonaqueous reaction medium for the production of esters based upon enzyme catalyzed reaction. Esterase or lipase enzymes remain active in the presence of silicone oil. Thus CalB, for example, may function as an enzyme in silicone oil serving as a nonaqueous reaction medium for the production of esters.

In one example, this approach was used with the addition of high-purity reactants in order to produce the desired ester. Specifically, ethyl alcohol (EtOH) and acetic acid (Ac), added to enzyme-sequestered silicone oil, allowed stable conversion of the water-labile ester products AcOEt and LacOEt.

Whether or not the esterification reaction itself actually occurs in silicone oil as a solvent, according to some embodiments silicone oil may serve as a distillation medium for water-soluble esters. Sample esters include but are not limited to miscible esters such as AcOEt, and partially soluble esters such as LacOEt.

The boiling point of silicone oil may be relatively high relative as compared to AcOEt or LacOEt, thereby facilitating separation of the product esters by simple distillation. As examples, poly-phenyl methyl siloxane oils are temperature stable to 250 degrees Celsius, while the dimethyl polymers are stable above 200 degrees Celsius. By contrast, the boiling points of AcOEt and LacOEt are lower: 77.1 degrees Celsius and 155 degrees Celsius, respectively.

Distillation of these ester products from their mixtures with silicone oils may thus be accomplished by simple one-step pot distillation. The ester product of interest becomes the purified vapor and ultimately, the condensate.

It is noted that separation of AcOEt by distillation from an aqueous solution is possible. However, separation of LacOEt from an aqueous reaction medium is only efficiently produced with more complex reactive distillation methods, increasing expense and lowering yield.

It is further noted that silicone oil can be employed in a simultaneous fermentation and ester synthesis process in order to avoid product inhibition of fermentation. Specifically, a fermentation process may be utilized to produce the alcohol comprising the starting material for the esterification reaction.

During fermentation, an overlay of silicone oil may provide both an oxygen barrier and a mechanism for selectively converting the products of fermentation (e.g., alcohol) to sequester the converted molecules in the oil layer.

As an example, during an organic acid fermentation, concentration of the desired acid product will increase and eventually become toxic to the fermentation organisms. Such accumulation of organic acid can cause that acidogenic microorganism to begin consuming the acid product, enter stationary phase, and thereby stall production. Or, the acid build-up may simply cause the acidogenic organism to die, halting the fermentation reaction.

Accordingly, in certain embodiments a layer of silicone oil including a reactive enzyme (e.g., Lipase B) may be added to the fermentation reaction mixture together with the second substrate alcohol. Then, the conversion and removal of the acid allows fermentation to continue, because the acid product is being continuously removed.

Moreover, a mixed fermentation may be designed using one or more fermenting microbes, wherein both the alcohol and organic acid reactants are produced. There, enzyme-charged silicone oil layer can remove both products as they are being generated.

Even if one or both of the reactants are only available at the silicone oil:fermentation broth interface, a solid-immobilized enzyme can be placed at that interface. This configuration allows conversion of reactants to product, avoiding potential for fermentation product inhibition.

Even where silicone oil is not utilized in the esterification reaction itself (e.g., esterification is performed with an acid-catalyzed reaction in aqueous or organic solvent), certain embodiments allow silicone oil to be used as an extraction medium. This approach leverages solubility of ester products in silicone oil, allowing their separation from a polar solvent.

For example, AcOEt may be produced by chemical or enzymatic methods in an aqueous or organic solution. Silicone oil present with the reaction mixture (e.g., as a layer) may specifically sequester all or a portion of the AcOEt product from the aqueous or organic phase.

By contrast, water and other components of the aqueous-organic solution will be rejected from the silicone oil. This technique affords a simple product purification step that separates the ester product from the underlying media.

If a desired product is only partially soluble in the silicone oil, this separation technique can be used in a continuous fashion to strip the desired product from the aqueous reaction layer. The resulting oil:product mixture can then be further distilled (as described above) in a batch or continuous operation.

Employing a separation technique utilizing silicone oil will allow batch or continuous collection of the desired ester product. And, once the ester product is removed from the silicone oil, recycling of the stripped silicone oil to the overlay is allowed.

### Example

A specific example of the use of silicone oil in the production of esters is now described. In this example, the organic acid was acetic acid (ACS grade) purchased from Amresco LLC of Cleveland, Ohio. The alcohol was produced from fermentation and distilled to 93 percent purity.

The silicone oil was PMX-200 silicone fluid available from Dow Corning Corp. of Auburn, Mich. The PMX-200 oil is a Poly dimethyl silicone oil with a viscosity of 10 centistokes at 25 degrees Celsius.

The Lipase B enzyme catalyzing the esterification reaction was provided physically immobilized in bead form as the NOVOZYM 435 product available from Novozymes of Denmark.

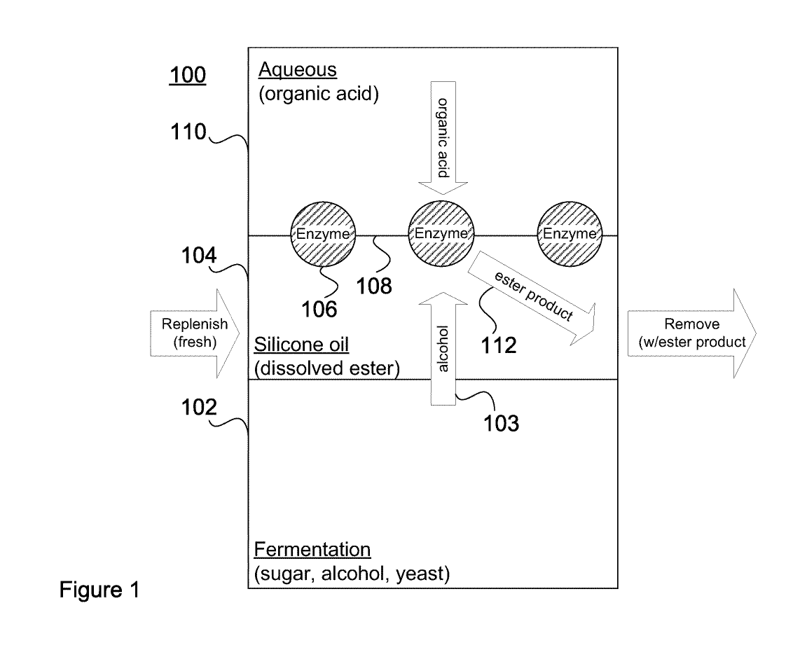

FIG. 1 is a simplified diagram showing the reaction configuration 100, which was conducted at 30 degrees Celsius. In particular, a bottom layer 102 comprised a mixture including sugars and a microbe (e.g., yeast) to perform the fermentation process producing the ethyl alcohol. As the less dense ethyl alcohol 103 was produced, it flowed through the intervening silicone oil layer 104 which included immobilized Lipase B beads 106 located at an interface 108 with the overlying aqueous mixture 110 including the organic acetic acid.

The enzyme-catalyzed reaction then occurred at the oil:aqueous interface, with the resulting ethyl acetate ester product 112 being soluble in the silicone oil. The solubility of ethyl acetate product partitioned between the aqueous layer and silicone oil layer, caused 30 percent of the ester product to be captured in the oil phase when equal volumes of aqueous and silicone oil partitions are used.

It is noted that the inherent buoyancy of the Lipase B immobilized as the NOVOZYM 435 product, naturally resulted in the beads residing at the silicone oil:aqueous interface. This is not required in all embodiments, however. Enzymes with different density properties could be employed to ensure maximum interaction between the enzyme, silicone oil, alcohol, or organic acid, or a combination of these.

For example, according to certain embodiments the physical interaction between one or more of the enzyme, the oil, and the aqueous material could be enhanced by creating an emulsion, thereby promoting interaction between the reaction components while still allowing the ester product to become solubilized in the silicone oil. For example, when using a reaction mixture containing an excess of Ethanol to Acetate (4:1) and a silicone oil phase of equal volume to the reaction mixture, with NOVOZYM 435, the resulting emulsion can be maintained by shaking at low rates. This emulsion produces a 50 percent product yield (measured against the limiting acetate) in 5 hours, and 30 percent of the ester product partitions in the oil phase. This 30 percent partition is dependent on the volume of the silicone oil phase.

FIG. 1 further shows flowing silicone oil including the dissolved ester product, from the reaction environment. Fresh silicone oil is in turn supplied in order to receive the additionally created ester product as it is generated.

While the above example utilized the combination of a particular organic acid (acetic acid), alcohol (ethyl alcohol), and silicone oil (poly-di-lmethyl), these particular constituents are not required. Different varieties of each component could be utilized in order to produce a desired ester product.

Examples of alcohols which could be selected as a starting material, can include but are not limited to: ethanol, isopropanol, propanol, butanol, isobutanol and higher alcohols.

Examples of organic acids which could be selected as a starting material can include but are not limited to acetic acid, hydroxypropanoic acid, lactic acid, succinic acid, butyric acid.

Examples of silicone oils which could be utilized as a reaction medium, can include but are not limited to: Poly dimethyl siloxane, and Poly methyl phenyl siloxanes of varying viscosities ranging from 10 centistokes (cSt) to over 2000 centistokes.

Examples of enzymes which could be utilized to catalyze esterification can include but are not limited to CalB, LipaseB, or other enzymes in EC 3.1.1.x.

In conformity with the hydrophobic, water-deficient environment offered by silicone oil as a reaction medium as described in these embodiments, useful enzymes may be expected to be present in halophilic microorganisms. Those organisms have evolved to conduct esterification in low water, high salt concentration environments.

Embodiments may find particular value in the generation of esters from large volumes of organic matter. For example, Arundo donax is an invasive reed that clogs waterways of the southwestern United States. That plant could be harvested in order to serve as a starting material for ester production.

Wood slag is another possible candidate as a starting material for ester production according to embodiments. That is, processes involving wood typically generate large amounts of waste material (e.g., sawdust, bark, and others) which are otherwise unused and must be disposed of. This wood slag is readily available in large quantities to serve as starting material for the generation of esters according to embodiments.

Organic matter by-product of certain agricultural activities, may comprise still other types of starting materials for ester production. For example, the edible almond kernels for sale in supermarkets, represent only a fraction of the mass of the almond fruit that is actually gathered from almond trees.

The remainder (e.g., hulls, shells, or others) of the collected fruit are discarded as almond husk waste product. Over many harvesting seasons, that almond husk waste product accumulates in large quantities or is disposed of by burning, or both, which can have adverse environmental impacts.

However, almond husk actually contains a significant amount of sugar (e.g., as much as 30 percent) that may be recovered in order to provide alcohol as a starting material for esterification.

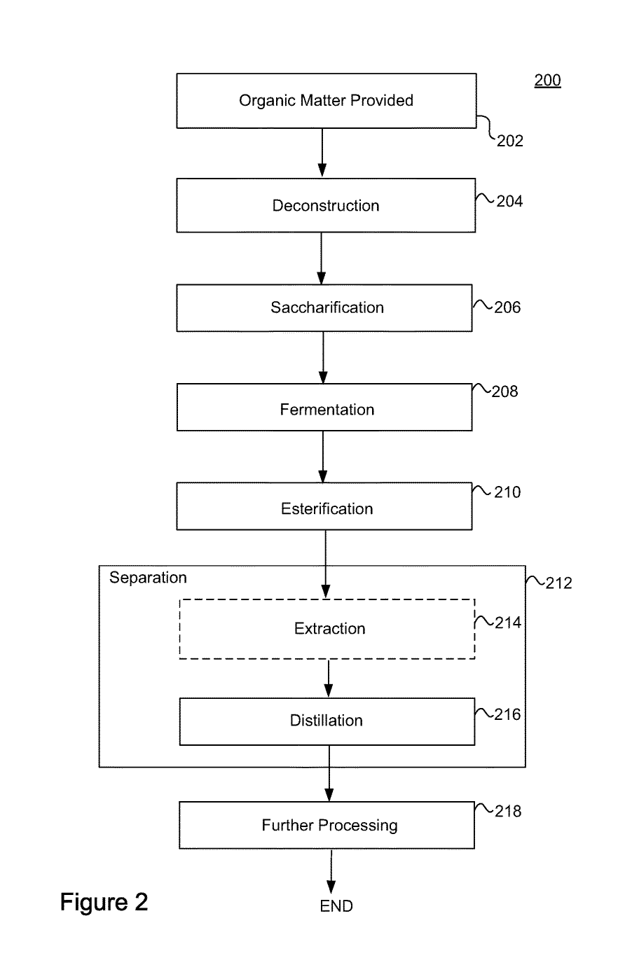

Accordingly, FIG. 2 is a simplified diagram showing a process flow 200 for the creation of esters from organic matter such as almond husks. In a first step 202, the organic matter is provided in its original form (e.g., almond husks, corn husks, apple cores, grape harvest detritus, others).

In a second step 204, the original organic matter is subjected to a process of deconstruction. This is a physical process of breaking up the organic matter into smaller pieces that are blended together in order to form a homogenous mixture serving as a starting material for the next step.

Specifically in a third, saccharification step 206, the deconstructed organic matter is converted into sugar according to an enzymatic or chemical process. Saccharification breaks down the organic matter from complex polymers (e.g., cellulose, lignin, or others), into simpler, individual sugar molecules or their small dimers or multimers (such as glucose, fructose, and xylose).

The saccharification process may be accomplished by the activity of microbes. These microbes include fungal species such as Trichoderma reesei and Aspergillus niger, archaeal species such as Halorhabdus utahensis or members of the Haloferax group, and members of the bacterial domain including Rhodothermus marinus and Thermus thermophilus. Saccharification may utilize the microbes themselves, or enzymes derived from these microbes, or enzymes derived from gene products of these microbes expressed in other organisms—Escherichia coli (E. coli), for example. Saccharification could also be accomplished by combining two or more of these approaches.

The next step 208 is fermentation. Here, the simple sugar molecules resulting from saccharification are converted into alcohol. As discussed above, this fermentation is accomplished by the activity of a microorganism (e.g., yeast). According to some embodiments the fermentation process may be a mixed fermentation process that also gives rise to the organic acid comprising the other starting material for the next step.

That next step 210 is the esterification reaction. This reaction utilizes the alcohol product of fermentation, an organic acid (which may also be produced by fermentation), and a catalyst as starting materials.

In certain embodiments, the esterification process may be enzyme-catalyzed. In some embodiments the esterification process may take place in silicone oil as a reaction medium.

The next step 212 is separation, wherein the ester product is separated from the reaction mixture. Where the reaction did not originally take place in silicone oil (e.g., acid-catalyzed aqueous esterification), the separation may comprise extracting 214 the ester into a silicone oil in which it is soluble, with other reaction components or side products remaining insoluble.

Whether the ester product is already present in silicone oil (e.g., as the original reaction medium), or is dissolved in the silicone oil by virtue of prior extraction, the separation process may further comprise distillation 216. There, the ester product is removed on the basis of having a lower boiling point relative to other components (including the silicone oil).

Further processing of the ester product is possible, as shown in step 218. Further removal of residual water can be accomplished using anhydrous salts or molecular sieves. While the above description has mentioned almond husks as suitable original organic matter, embodiments are not limited to this particular starting material. Other organic matter suited to provide alcohol (e.g., by way of saccharification or fermentation) as a starting material for esterification, can include but are not limited to corn husks, apple pomace, wood waste, paper towels, newspaper, and pistachio hulls.

While the above description has focused upon the use of silicone oil for extraction of ester products, silicone oil may also be used in the extraction of medium chain length alcohols (e.g., butanol) from a polar solvent (such as water). For example, during butanol synthesis performed by a microorganism, butanol can become toxic at concentrations above 1 percent. Beyond that concentration, further microbial fermentation requires ongoing product extraction in order to continue.

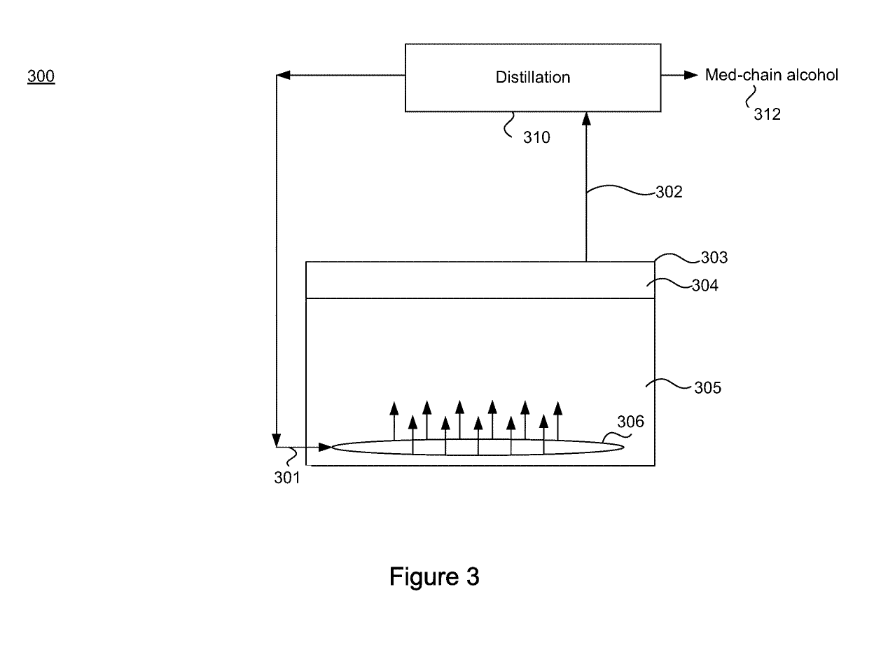

Accordingly, FIG. 3 shows a simplified view of a reaction environment 300 according to an alternative embodiment. Here, silicone oil functions as a mobile extraction medium for medium chain length alcohol.

Silicone oil is cycled through the fermentation broth 305, producing a layer 304 that retains the medium-chain alcohol while efficiently excluding water. Specifically, oil is cycled through inlet port 301, becomes distributed through the aqueous fermentation broth by element 306 (such as a diffuser), with the oil forming a product-oil layer 304 above the aqueous phase.

The silicone oil is then cycled through output port 302 to a simple vacuum distillation apparatus 310 for removal of the medium chain length product 312. Following distillation, the oil is then cycled back to the input port 301.

When using silicone oil as a mobile phase, the distillation may be a simple distillation, where the boiling point of silicone oil is significantly higher than that of the product. As an example, butanol boils at 117.7 degrees Celsius while typical silicone oils boil above about 250 degrees Celsius.

While the above description has focused upon extraction of butanol as an exemplary medium chain length alcohol, embodiments are not limited to this particular product. Examples of other forms of medium chain length alcohols that may be formed by microbial fermentation (and hence suitable for extraction using silicone oil), can include but are not limited to alcohols with between about 4-10 carbons in either a linear or branched chain configuration. Examples include but are not limited to butanol, isobutanol, pentanol, hexanol, heptanol, octanol, and others.

And while the above description has mentioned extraction of medium chain length alcohols from water, embodiments are not limited to that particular solvent. Other types of polar solvents may be compatible with fermentation processes. Examples can include but are not limited to short alcohols (such as methanol and ethanol), as well as short organic acids (such as acetate).

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

The invention claimed is:

1. A method comprising:
conducting a reaction between an alcohol and an organic acid to produce an ester; and
dissolving the ester in a silicone oil.

2. A method as in claim 1 wherein the reaction is conducted in the silicone oil.

3. A method as in claim 2 further comprising catalyzing the reaction with an enzyme.

4. A method as in claim 3 wherein the enzyme is produced by a halophilic organism.

5. A method as in claim 4 wherein the enzyme is Lipase B.

6. A method as in claim 3 wherein the enzyme is immobilized.

7. A method as in claim 6 wherein the enzyme is immobilized at an interface between the silicone oil and an aqueous mixture including the organic acid.

8. A method as in claim 2 further comprising:
removing the silicone oil including the dissolved ester; and
replenishing fresh silicone oil.

9. A method as in claim 2 further comprising simultaneously performing a fermentation process to produce the alcohol.

10. A method as in claim 9 wherein:
the fermentation process is simultaneously performed in a same reaction vessel containing the silicone oil; and
the alcohol flows into the silicone oil to drive the reaction.

11. A method as in claim 9 wherein the fermentation process utilizes an agricultural waste product as a starting material.

12. A method as in claim 9 wherein the fermentation process is a mixed fermentation process also producing the organic acid.

13. A method as in claim 1 wherein the ester is dissolved in the silicone oil by an extraction process.

14. A method as in claim 13 wherein the reaction is catalyzed by an acid.

15. A method as in claim 13 wherein the reaction is catalyzed by an enzyme.

16. A method as in claim 1 further comprising separating the ester from the silicone oil by distillation.

17. A method as in claim 1 wherein the reaction occurs in an emulsion comprising the silicone oil and an aqueous phase including the organic acid.

18. A method comprising:
conducting a microbial fermentation reaction to generate a medium chain length alcohol;
dissolving the medium chain length alcohol in a silicone oil; and
removing the medium chain length alcohol from the silicone oil by distillation.

19. A method as in claim 18 further comprising:
following removal of the medium chain length alcohol from the silicone oil, circulating the silicone oil back to the microbial fermentation reaction to dissolve additional quantities of the medium chain length alcohol.

20. A method as in claim 18 wherein the medium chain length alcohol comprises butanol.
